(19) United States
(12) Patent Application Publication
(10) Pub. No.: US 2008/0287942 A1
(43) Pub. Date: Nov. 20, 2008

(54) RF Ablation Catheter with Side-Eye Infrared Imager

(76) Inventor: David C. Amundson, Boulder, CO (US)

Correspondence Address: PETER F WEINBERG;GIBSON DUNN AND CRUTCHER LLP, SUITE 4100, 1801 CALIFORNIA STREET, DENVER, CO, 80202, US

(21) Appl. No.: 11/750,251

(22) Filed: May 17, 2007

(51) Int. Cl. A61B 18/14

(52) U.S. Cl. 606/41

(57) ABSTRACT

An ablation catheter has an imager useful in connection with an ablation operation such as used in the treatment of a cardiac arrhythmia. The catheter has an ablation electrode positioned on a catheter tip, and the imager is located on a side of the catheter. Because the catheter tip is generally articulated when the lesion is formed, this position of the imager provides an image that corresponds with the position of the catheter when it is used to form a lesion. Rings for recording tissue electrograms are also positioned on the catheter tip. This arrangement allows the user to accurately place and contact the electrode and to monitor the ablation process such as by viewing bubbles and thrombi that may undesirably be formed.

### BACKGROUND

In the field of endocardial RF catheter ablation of cardiac arrhythmias, an electrode on the distal end of a catheter is placed on endocardial tissue and RF energy is applied to the tissue to ablate a critical piece of tissue responsible for maintaining the arrhythmia. Usually, the culprit tissue is only recognizable from its electrical potential characteristics. Sometimes, ablations are made using anatomical criteria for the ablation pattern. In atrial flutter or atrial fibrillation ablations, a contiguous line or circle is required to terminate the arrhythmia.

The basic goal of RF ablation is to make a substantial lesion (100-600 cubic mm depending on electrode size and flow rate at the ablation site) and to avoid complications (thrombi, perforation, injuring heart structures or nearby non-heart structures). Critical to the success of the ablation are:
(1) For anatomical ablations such as atrial flutter or some pulmonary vein isolation procedures, placing the electrode on the appropriate tissue. For example, in a pulmonary vein (PV) isolation procedure, the physician endeavors to ablate a certain distance away from the pulmonary vein ostia. Failure to do so may result in stenosis of the PV;
(2) Tissue contact with a substantial part of the electrode portion against tissue. This is generally accomplished by placing the long end of the electrode flush against tissue, if possible. This technique has been shown to create the largest lesions and avoid complications such as perforation and thrombi production
(3) Not producing thrombi;
(4) Not producing bubble showers; and
(5) Not producing steam pops. The effect of these issues on ablation is as follows:

| | |
|---|---|
| Placement | In anatomical ablations such as Aflutter or PV isolation, poor placement can lead to |
| | complications such as PV stenosis or incontiguous lesions and procedure failure |
| Contact | Largest lesions produced with entire side of electrode contacting tissue. More |
| | perpendicular contact against tissue will produce smaller lesions. If the proximal end is in |
| | poor contact stagnant blood can be present at the very hottest point of the electrode |
| | (proximal end) and can lead to thrombi production |
| Thrombi | In right heart, thrombi production can lead to electrode adhesion to the endocardial surface |
| | and removal can cause perforation. In left heart, thrombi can cause strokes and |
| | myocardial infarcts. Thrombi generally occur in stagnant local flow areas, particularly in |
| | the setting of high wattage ablations. |
| Bubbles | Classified as Type I: occasional bubble and Type II: bubble showers. Experts differ on |
| | Type 1 bubbles: some consider it a marker to cease ablation, others consider it a sign a |
| | good ablation is occurring. Type II bubbles are to be avoided and the ablation should be |
| | terminated. Type II bubbles are regarded by some as a precurser to steam pops. |
| Steam pops | Steam pops occur when the hottest part of tissue about one mm from the electrode |
| | explodes because the temperature has reached 100 deg C. at that point. The intervening |
| | tissue between electrode and hottest tissue point usually perforates, leaving a cratered |
| | appearance in the lesions.. Steam pops can result in heart wall perforation. |

All of these issues are difficult to evaluate with the current imaging technologies: fluoroscopy, intracardiac ultrasound (ICE) and navigational systems (e.g. CARTO™, EnSite™). Fluoroscopy gives a global view of the approximate position of the ablation catheter relative to the heart silhouette. Issues 2-5 are not observed by fluoroscopy. ICE also gives approximate location of the ablation catheter to various cardiac structures observable in ultrasound. ICE can also detect steam bubbles once they reach a certain size and only if the ICE catheter is oriented in a plane to observe them. The navigational systems provide the most accurate tissue locater relative to cardiac structures and is the system of choice for complicated ablations such as pulmonary vein isolation, but like fluoroscopy does not address issues 2-5.

| Technology | Placement | Contact | Thombi | Bubbles | Steam pops |
|---|---|---|---|---|---|
| Fluoroscopy | Global | No | No | No | No |
| ICE | Proximity to | Occasionally, | No | Yes, if large | No |
| | viewable cardiac | but ICE | | enough and if | |
| | structures such as | catheter and | | catheter is in | |
| | CS, PV's, tricuspid | ablation | | bubble plane | |
| | valve, mitral valve | catheter need | | | |
| | | to be in | | | |
| | | favorable | | | |
| | | positions | | | |
| Navigational | Accuracy to about 1 cm | No | No | No | No |
| | from mapped | | | | |
| | structure | | | | |

### INVENTION SUMMARY

Disclosed is a novel infrared imager which views out of the side of the catheter and located proximal to the proximal end of the ablation electrode, which addresses these concerns. The infrared imaging system has been previously disclosed in U.S. Pat. No. 6,178,346, and in Patent App 10912732 (both of which are incorporated herewith by reference) but differs from these patents in that the imager is located on the side of the catheter. Illuminating and received light are both bent about 90 deg by a prism located at the distal end of the optical assembly. The imager is positioned on the catheter so that when the catheter is articulated 180 deg, the imager is pointed outwards.

Imaging to the side is advantageous in catheter ablation because the physician endeavors to place the electrode on its side to produce an effective ablation. Generally it is articulated 90-180 deg so that the electrode long axis is parallel to the target heart, tissue.

In that position, the side-eye imager is directly viewing the target tissue site and the infrared images can be used to effect “a landing” of the electrode against the tissue while under continuous view of the real-time side-eye imager. In forward-viewing systems, the catheter tip needs to be pointed at the target tissue, followed by an articulation to aim the electrode side-on against tissue. The articulation prevents the possibility of viewing the electrode “landing” on tissue.

Imaging to the side is also advantageous in viewing lesions and in making a contiguous lesion. A forward-viewing catheter making a lesion on the electrode side needs to be rotated about 90 deg to view an ablation. The articulation and manipulation of this maneuver usually displaces the tip far away from the ablation site. In contrast, the side-eye imager is close enough to the electrode proximal end to view some of the lesion production. Sliding the catheter forward a few mm will place the lesion in full view of the side-eye imager and the electrode can be fine-positioned relative to this lesion to make a second connecting lesion contiguous with the first lesion, while maintaining the same articulation. Alternatively, the catheter can be dragged forward and as the catheter is dragged, lesion creation can be verified with the side-eye imager.

The advantages in the above five mentioned areas are:
1. Electrode Placement.
   When the catheter is articulated about 180 deg and about to be placed on tissue, the tissue is directly imaged be the side-eye imager. The physician can fine-tune the electrode position relative to structures such as the pulmonary vein
2. Electrode Contact.
   When the electrode is placed flush against tissue, the side-eye imager will also be against tissue. Because of the sharp rise of signal intensity when touching tissue, touching is readily apparent in the infrared image and significantly more detail is apparent. Alternatively, a graphical guide could be presented on the imaging screen to indicate the proximity to tissue of the side-eye imager. This would be very beneficial since flush contact with the long axis of the electrode produces the largest lesions and the least complications.
3. Thrombi
   Thrombi generally occur at the proximal end of the ablation electrode since this is the site of highest temperature and the current density change is largest at this point. Having flush contact minimizes thrombi production, while poorer contact when the proximal end of the ablation electrode has a separation between tissue can be an area of poor flow and exposure of red blood cells to the thrombis-producing portion of the electrode. Moreover, if thrombi form they will be imaged by the nearby side-eye image. If the imager views thrombi, it would be reasonable to cease the ablation. In summary, the side-eye imager provides feedback on how flush the contact area is and additionally can view thrombi production if it occurs and therefore terminate the ablation.
4. Bubbles
   The presence of Type II bubbles (bubble shower) is used by some clinicians as a “fuse” to terminate the ablation (K Kuch et al. Circulation). The only means today of detecting bubbles is with the use of an ICE catheter. If the ICE catheter is oriented in the plane of the bubbles, they will not be seen.
   also the bubbles need to achieve a certain size to viewable with an ICE catheter. The side-eye imager being located next to the proximal end of the ablation electrode is in a ideal position to view bubbles, since the electrode end is the most likely place for them to occur. Unlike ICE, the imager need no be in a particular orientation and can see bubbles as small as the resolution limits of the imaging system (20 microns). Moreover, bubbles are highly reflective in infrared endoscopy, often having twice the signal intensity as a nearby heart structure. There easy visual detection indicates the bubble detection could be detected by the imaging system resulting in immediate cessation of the ablation.
5. Steam Pops
   Steam pops occur approximately 1 mm into the tissue (the hottest point) when the temperature exceeds 100 deg C., resulting in bursting of the tissue. They can lead to perforation. Steam pops are generally proceeded by bubble showers, thrombi formation and are more likely to occur near the electrode's hottest point, the proximal end of the electrode. Monitoring this region with the side-eye imager and being able to detect the precursors of steam pops permits the ablation to be terminated prior to any steam pops.

In addition, the side-eye imager experiences less whip from heart motion than does a forward-viewing catheter. The greatest whip due to heart motion occurs on the distal end of the catheter. More proximal positions will experience less whip, depending on the articulation, with the least whip occurring when the catheter is 90-180 deg articulated. Additionally, an articulated catheter with a side-eye imager could be positioned to further minimize the motion created by heart contraction, since the articulated portion is often in contact with heart wall, which provides additional stability to heart motion. If the distal end is also contacting tissue the side-eye imager is stabilized on both ends.

Lastly, correlating the infrared images to the fluoroscopic position of the catheter is superior for the side-eye imager over the forward-viewing catheter. With a forward-viewing catheter, it is difficult to discern where the tip is pointing on fluoroscopy. Moreover the up-down, L-R orientation is always changing depending on how the operator is rotating the handle. Fluoroscopically, it is simple to know where the side-eye imager is located and therefore where it is pointing: It is pointing to the outside of an articulated catheter and between the tip and the first recording ring. If, for example the image bundle were oriented so that up is the ablation electrode and down is towards the first recording ring, then the orientation of the image can be discerned from observing the catheter on fluoroscopy. As the catheter is observed in the usual articulated condition, the physician can point the side-eye imager at the expected position of various cardiac features. Up-down, L-R are determined from the orientation of the catheter in the fluoroscopic image. The image is between the ablation tip and the first recording ring, up is towards the electrode and down is towards the ring.

### FIGS.

1. Shows the distal end of the ablation electrode.

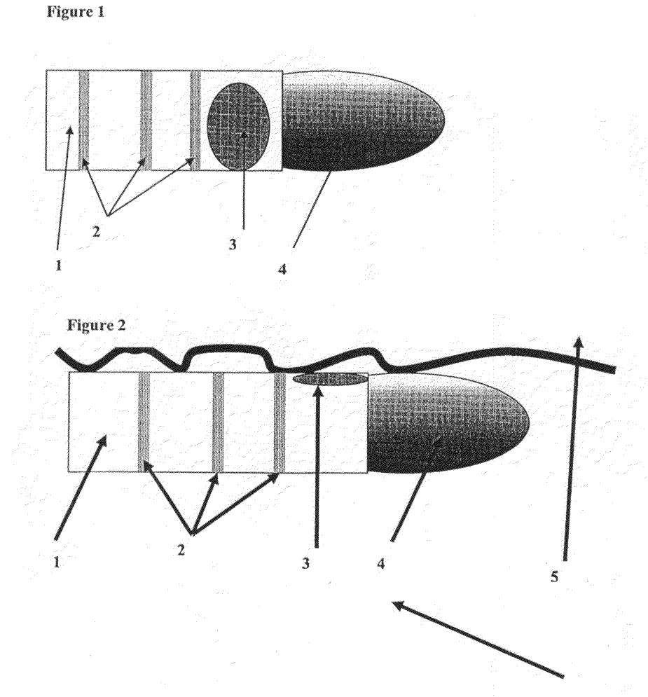

2. Shows the distal end against tissue and rotated about 90 deg from FIG. 1.

3. Shows the internal assembly of the catheter and the light path through the prism out the side of the catheter.

4. (A) The catheter is articulated and the side-eye imager is viewing a pulmonary vein (PV). (B) The infrared image of the PV

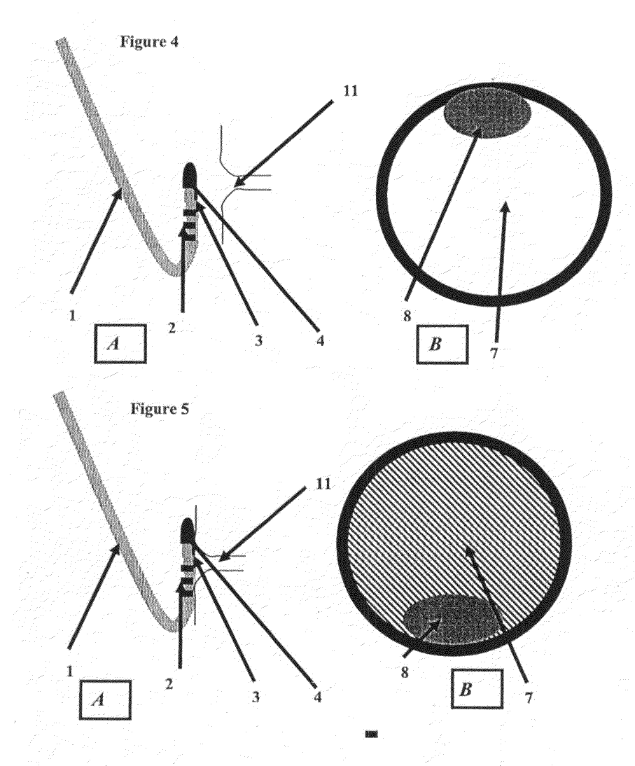

5. (A) The catheter is articulated and the side-eye imager is touching the periosteal tissue near the PV and has been positioned higher on the figure than in FIG. 4(B) The infrared image showing tissue contact.

6. (A) The catheter is articulated and the side-eye imager is touching heart wall tissue and forming a lesion. (B) The infrared image showing tissue contact and the lesion produced.

7. (A) The catheter has been moved forward a few millimeters and is forming a second lesion. (B) the infrared image showing tissue contact, the old lesion and the new lesion produced.

8. (A) The catheter is articulated and the side-eye imager is touching the tissue. (B) The infrared image showing bubble formation

9. (A) The catheter is articulated and the side-eye imager is touching the tissue. (B) The infrared image showing thrombus production.

### DETAILED EMBODIMENTS

This device operates with the infrared imaging system as described in U.S. Pat. No. 6,178,346. As seen in FIG. 3, the imaging system has been modified through the use of a prism (20) which directs both illumination and returned reflected light in about a 90 deg angle to permit viewing on the side of the catheter.

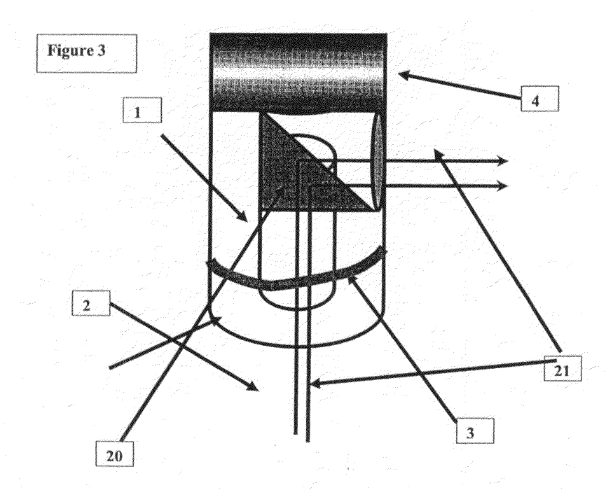

As seen in FIG. 1, the ablation catheter (1) contains three rings (2) to record electrograms of underlying tissue. Between the distal end of the electrode (4) and the first ring is located the side-eye imager (3). In FIG. 2, the catheter is rotated about 90 deg and placed against tissue (5).

As the side-eye imager is placed against tissue, the signal amplitude returning to the imaging bundle will dramatically increase. This will be seen on the infrared image as a high-detail image. Alternatively, the signal count could be used to construct a graphic, such as a bar which goes up to maximal level once the side-eye imager is seated on tissue. Since signal counts can be between 2000-3000 counts when touching and 500-1000 when a few mm away, this is a sensitive indicator regarding distance to tissue.

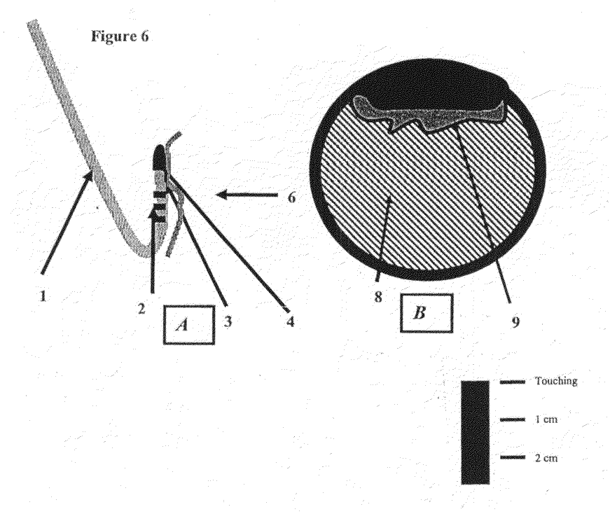

FIG. 4 shows the catheter (1) articulated and a 2-3 mms away from a PV ostium. The side-eye imager (3) located between the electrode and first recording ring (2) is now pointed at the PV. As seen in the image (7) from the side-eye image, the PV (8) is located at the top of the screen since it is closer to the electrode tip than the first recording ring. As the physician places the ring safely on the PV sleeve (FIG. 5), the PV os (8) now is at the bottom of the screen (7), since the PV os is closer to the first recording ring. In addition, the tissue image shows detail associated with the side-eye imager touching tissue. This assures that the proximal end of the ablation electrode is also lying flush against tissue, thereby improving the chances of a large and safe lesion.

FIG. 6 shows the catheter (1) touching another endocardial wall (6). Again the tissue appearance has much detail resulting from high signal count indicating the side-eye imager (3) is touching tissue. In this embodiment, to the left of the infrared image (7) contains a bar which indicates distance of the side-eye imager from tissue. based on the signal count. As shown in FIG. 6, the side-eye imager is now touching the tissue.

An ablation is made (9) and is depicted in the FIG. 6 as a black spot with lighter edges in an irregular fashion. The lesion grows from the top of the screen during the ablation since up is defined as towards the ablation electrode and down is towards the first recording ring.

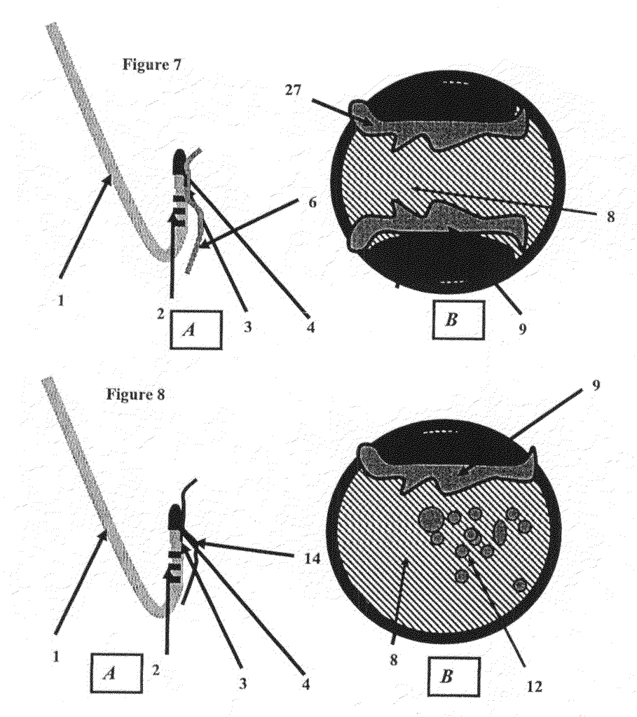

In FIG. 7, the electrode is advanced a few millimeters higher as seen on the figure. Now the first ablation (9) appears at the bottom of the screen since it is now closer to the first recording ring. A second ablation occurs (27) growing from the top of the screen from the ablation electrode. As the lesion is observed connecting with the first lesion (9), a contiguous lesion is formed and the ablation can be terminated.

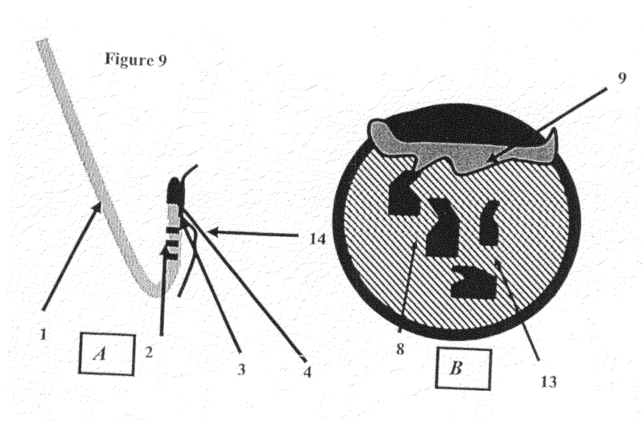

FIGS. 8 and 9 demonstrate how the presence of bubble formation and thrombi would appear in the image. In FIG. 8, the ablation catheter (1) is creating a lesion (9) when bubbles (12) appear in the image (8). Bubbles are associated with high signal count and are easily observable. When the bubbles approach “bubble shower status” (Type II bubbles) the physician can manually terminate the ablation. Alternatively, the bubbles can be detected by image processing features and the ablation can be automatically terminated.

FIG. 9 shows the ablation catheter (1) creating a lesion (9) when thrombi (13) appear in the image (8). The appearance of thrombi would suggest to the physician that the ablation should be terminated.

Summary

Disclosed is an ablation catheter with an infrared imager on the side of the catheter between the ablation electrode and the first recording ring. The advantages of the side-eye imager are as follows:

Placement

The side-eye imager can effect a “landing” on the appropriate tissue segment. From the time the physician articulates the catheter so as to place the long end of the electrode against tissue to actually touching tissue, the side-eye imager is displaying in real time the entire process, permitting placement fine tuning.

Contact

Contact of the proximal end of the ablation electrode is assured by the high signal count, detailed image from the side-eye imager. There is no other methodology which can consistently inform the physician about electrode contact like the side-eye imager. This improves the chances of a large and safe lesion.

Bubbles

Bubbles are easily detected by the side-eye imager and gives feedback about when to terminate the ablation.

Thrombi

Thrombi are typically produced at the proximal end of the ablation electrode where current densities and temperature are highest, a few mm from the side-eye imager. Observance of thrombi provides important feedback to the physician who will likely terminate the ablation at that point.

Image Stability

Since the side of the catheter has less whip than a forward-viewing catheter, the image stability is improved accordingly.

Fluoroscopic Verification of Infrared Image

Since the side-eye imager is located between the ablation electrode and the first recording ring and facing outward when articulated, the direction of the side-eye imager is easily determined from fluoroscopy. Moreover, image orientation is set with UP towards the ablation electrode, DOWN towards the first recording ring.

Lesion Viewing

Since the lesion extends several mm from the electrode, the lesion will be seen growing from the top of he screen and growing in a downwards direction. The entire lesion can be imaged by simply sliding the catheter in a forward direction for a few mms.

Contiguous Lesions

Contiguous lesions can be formed by sliding the catheter forward a few mms and watching the new lesion grow downwards and connect with the first lesion at the bottom of the screen. If the catheter is dragged in a forward direction, the lesion will be imaged immediately after it has been formed.

Universality

The side-eye imager can be applied to any RF ablation catheter whether for example 4 mm or 8 mm, irrigated or non-irrigated, current or future ablation electrode.

What is claimed is:

1. A medical instrument comprising:
a catheter having an axis and a side, the catheter having an articulatable tip region;
means for ablation positioned on the tip region;
an imager positioned on the tip region proximal to the means for ablation, wherein the imager is positioned on the catheter side and is capable of viewing in a side direction that is perpendicular to the catheter axis.

2. The instrument of claim 1, further comprising means to record an electrogram of a patient.

3. The instrument of claim 2, wherein the means to record an electrogram are on the tip region.

4. The instrument of claim 1, wherein the imager produces a signal having an amplitude, and the signal amplitude significantly increases when the imager is placed against a tissue of the patient.

5. The instrument of claim 1, wherein the imager produces a signal having an amplitude, and wherein the instrument displays an indicator related to the distance between the imager and a tissue of the patient.

6. The instrument of claim 1, wherein when the articulatable tip region is articulated within the vasculature of a patient, the imager can view a tissue of a patient when it is sufficiently close to the tissue.

7. The instrument of claim 1, wherein the imager can image a pulmonary vein.

8. The instrument of claim 7, wherein the imager can image a pulmonary vein relative to the means for ablation or to the means to record the electrogram of the patient.

9. The instrument of claim 1, wherein the imager can image a bubble associated with an operation of the means for ablation.

10. The instrument of claim 1, wherein the imager can image a thrombus associated with an operation of the means for ablation.

11. The instrument of claim 1, wherein the instrument produces an image associated with the imager having directions such that a direction toward the means for ablation is opposite from a direction toward the means to record an electrogram of a patient.

12. The instrument of claim 1, wherein the instrument produces an image associated with the imager having directions such that an up direction is toward the means for ablation and the down direction is toward the means to record an electrogram of a patient.

13. The instrument of claim 1, wherein the means for ablation can form a lesion on a tissue of a patient and the imager can image the lesion.

14. The instrument of claim 13, wherein the catheter can be moved to make a contiguous linear lesion, and the imager can image the contiguity of the lesion.

15. The instrument of claim 13, wherein the catheter can be moved to make a contiguous series of spot lesions, and the imager can image a border of the lesions.

16. The instrument of claim 13, wherein the instrument can be viewed with fluoroscopy to determine an orientation of the imager.

17. A method of deploying an ablation catheter comprising the steps of:
inserting an ablation catheter into a vasculature of a patient;
deflecting an catheter tip, wherein the tip has a means for ablation;
imaging a tissue of a patient with an imager on the catheter tip, wherein the imager images in a side direction relative to a catheter axis; and
using the means for ablation.

18. The method of claim 17, further comprising means to record an electrogram.

19. The method of claim 18, wherein the means to record an electrogram are situated on a portion of the catheter tip that can be deflected.

20. The method of claim 17, wherein the imager produces a signal having an amplitude, and the signal amplitude significantly increases when the imager is placed against a tissue of the patient.

21. The method of claim 17, wherein the imager produces a signal having an amplitude, and wherein the instrument displays an indicator related to the distance between the imager and a tissue of the patient.

22. The method of claim 17, wherein when the tip is deflected within the vasculature of a patient, the imager can view a tissue of a patient when it is sufficiently close to the tissue.

23. The method of claim 17, wherein the imager can image a pulmonary vein.

24. The method of claim 23, wherein the imager can image a pulmonary vein relative to the means for ablation or to the means to record the electrogram of the patient.

25. The method of claim 17, wherein the imager can image a bubble associated with an operation of the means for ablation.

26. The method of claim 17, wherein the imager can image a thrombus associated with an operation of the means for ablation.

27. The method of claim 17, wherein the instrument produces an image associated with the imager having directions such that a direction toward the means for ablation is opposite from a direction toward the means to record an electrogram of a patient.

28. The method of claim 17, wherein the instrument produces an image associated with the imager having directions such that an up direction is toward the means for ablation and the down direction is toward the means to record an electrogram of a patient.

29. The method of claim 17, wherein the means for ablation can form a lesion on a tissue of a patient and the imager can image the lesion.

30. The method of claim 29, wherein the catheter can be moved to make a contiguous linear lesion, and the imager can image the contiguity of the lesion.

31. The method of claim 29, wherein the catheter can be moved to make a contiguous series of spot lesions, and the imager can image a border of the lesions.

32. The method of claim 29, wherein the instrument can be viewed with fluoroscopy to determine an orientation of the imager.
